(19) United States
(12) Patent Application Publication
(10) Pub. No.: US 2015/0224031 A1
(43) Pub. Date: Aug. 13, 2015

(54) MICRO-BOLUS FEEDER KIT FOR INFANTS

(71) Applicant: NADINE M. METHNER, WILLIAMSTON, MI (US)

(72) Inventor: NADINE M. METHNER, WILLIAMSTON, MI (US)

(21) Appl. No.: 14/602,253

(22) Filed: Jan. 21, 2015

Related U.S. Application Data
(60) Provisional application No. 61/937,872, filed on Feb. 10, 2014.

(51) Int. Cl. A61J 15/00; A61J 11/00; A61M 5/00; A61J 9/00; A61M 5/31

(52) U.S. Cl. CPC A61J 15/0011; A61J 15/0003; A61J 9/00; A61J 15/0026; A61M 5/3135; A61M 5/002; A61J 11/0005; A61J 2200/70; A61J 2009/0669

(57) ABSTRACT

The micro-bolus feeder kit for infants includes at least one syringe and at least one adapter fitting for connecting a nipple ring (or nipple collar) and a nipple to the syringe so that the syringe may be used to deliver metered amounts of milk, formula, or other liquid nutrition to the infant. The syringe is preferably a 3 mL to 5 mL syringe with a plunger and graduated markings to show the volume of liquid in the syringe, and is capable of delivering the liquid in one-tenth to two-tenths milliliter increments. The adapter fitting is an annular disk adapted for gripping the tubular syringe barrel by friction or pressure fit, or resiliently, and has external threads on its periphery dimensioned and configured for receiving a nipple ring in order to attach a nipple to the delivery end of the syringe.

### CROSS-REFERENCE TO RELATED APPLICATION

This application claims the benefit of U.S. Provisional Patent Application Ser. No. 61/937,872, filed Feb. 10, 2014.

### BACKGROUND OF THE INVENTION

1. Field of the Invention

The present invention relates to infant feeding, and particularly to a micro-bolus feeder kit for infants.

2. Description of the Related Art

Dysphagia (i.e., difficulty in swallowing) is common in premature infants. Typically, such infants are fed thickened fluids. The thicker consistency makes it less likely that an infant with dysphagia will aspirate while they are drinking. A range of commercial thickening agents are available for purchase for the dietary management of dysphagia. However, there are concerns about the effects of commercial thickeners on infants' intestinal and digestive systems. Thus, there is a need to provide alternatives so that infants can be fed small amounts of food without additional thickening agents. Further, it is obviously desirable to minimize stress on a medically fragile infant, such as a premature infant undergoing his or her first nutritive sucking experience, or infants suffering respiratory compromise or with a history of neurologic impairment.

Thus, a micro-bolus feeder kit for infants solving the aforementioned problems is desired.

### SUMMARY OF THE INVENTION

The micro-bolus feeder kit for infants allows for the measured and controlled feeding of liquid nutrients to infants. The kit includes at least one oral syringe and at least one adapter fitting for connecting a nipple ring (or nipple collar) and a nipple to the syringe so that the syringe may be used to deliver metered amounts of milk, formula, or other liquid nutrition to the infant. The syringe is preferably a 3 mL to 5 mL syringe with a plunger and graduated marking to show the volume of liquid in the syringe, and is capable of delivering the liquid in one-tenth to two-tenths milliliter increments. The adapter fitting is an annular disk adapted for gripping the tubular syringe barrel by friction or pressure fit, or resiliently, and has external threads on its periphery dimensioned and configured for receiving a nipple ring in order to attach a nipple to the delivery end of the syringe. The kit may contain both 3 mL and 5 mL disposable syringes and a single adapter fitting adapted for attachment to either size syringe, or different size adapters for the different size syringes. Alternatively, the disposable syringe and adapter fitting may be manufactured integrally; i.e., as one piece. The kit may optionally include a nipple ring threadable to the adapter fitting and adapted to attach any nipple having a conventional annular flange to the syringe, and also may optionally include different types of nipples.

These and other features of the present invention will become readily apparent upon further review of the following specification and drawings.

### BRIEF DESCRIPTION OF THE DRAWINGS

FIG. 1 is an environmental, perspective view of a micro-bolus feeder kit for infants according to the present invention.

FIG. 2 is a partially exploded environmental perspective view of the micro-bolus feeder kit for infants according to the present invention.

FIG. 3 is a perspective view of a micro-bolus feeder for infants according to the present invention.

FIG. 4 is a front view of a micro-bolus feeder kit for infants according to the present invention.

Similar reference characters denote corresponding features consistently throughout the attached drawings.

### DETAILED DESCRIPTION OF THE PREFERRED EMBODIMENTS

The micro-bolus feeder kit for infants allows for the measured and controlled feeding of liquid nutrients to infants. The kit includes at least one oral syringe 14 and at least one adapter fitting 16 for connecting a nipple ring 42 (or nipple collar) and a nipple 12 to the syringe 14 so that the syringe 14 may be used to deliver metered amounts of milk, formula, or other liquid nutrition to the infant. The syringe 14 is preferably a 3 mL to 5 mL syringe with a plunger and graduated markings to show the volume of liquid in the syringe 14, and is capable of delivering the liquid in one-tenth to two-tenths milliliter increments. The adapter fitting 16 is an annular disk adapted for gripping the tubular syringe barrel 20 by friction or pressure fit, or resiliently, and has external threads 34 on its periphery dimensioned and configured for receiving a nipple ring 42 in order to attach a nipple 12 to the delivery end of the syringe 14. The kit may contain both 3 mL and 5 mL disposable syringes and a single adapter fitting adapted for attachment to either size syringe, or different size adapters for the different size syringes. The kit may optionally include a nipple ring threadable to the adapter fitting and adapted to attach any nipple having a conventional annular flange to the syringe, and also may optionally include different types of nipples.

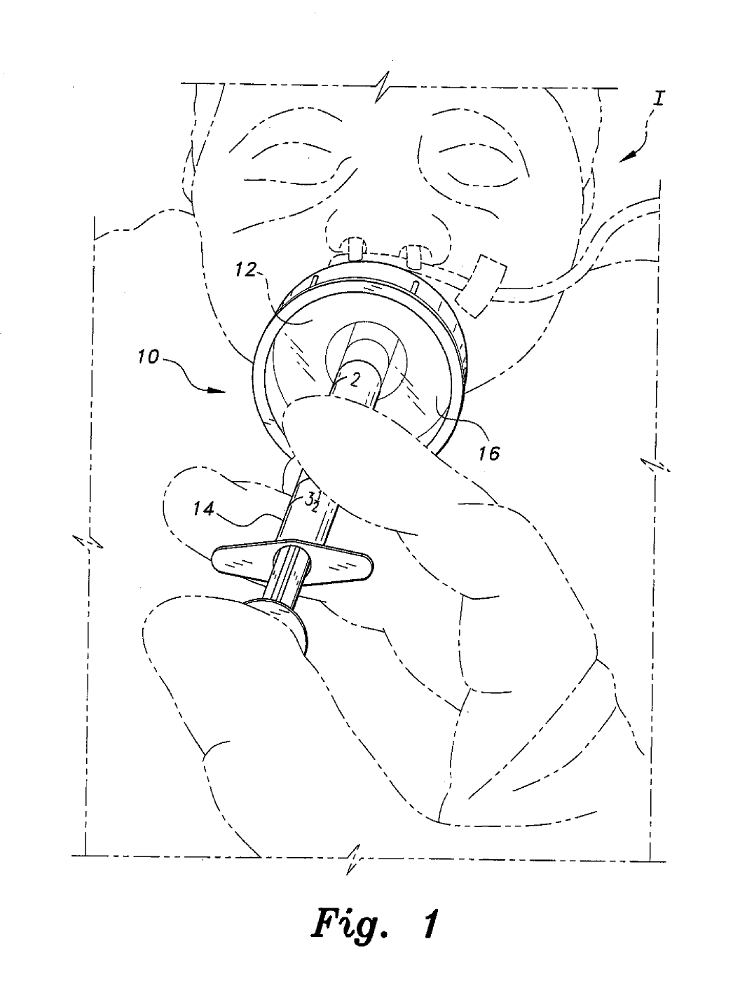

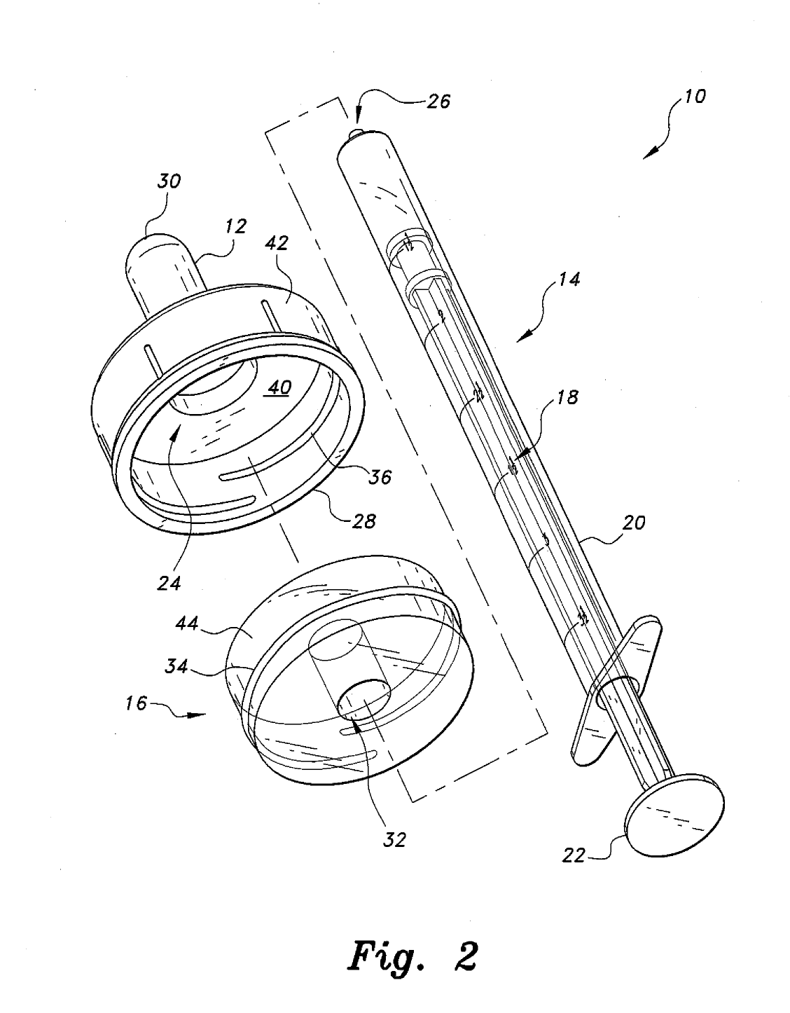

Referring to FIGS. 1 and 2, a micro-bolus feeding system 10 for infants allows for the measured and controlled feeding of liquid nutrients to infants, such as exemplary infant I. The system 10 includes an elastomeric infant feeding nipple 12 similar to that found in conventional baby bottles and the like. It should be understood that the nipple 12 shown in FIGS. 1 and 2 is shown for exemplary purposes only, and that the nipple 12 may be any suitable type of artificial nipple or teat. Additionally, although the system 10 is shown as having an attachment portion 28 including a flange 40 and a nipple ring 42, it should be understood that the basic micro-bolus feeder kit need not include the attachment portion 28. The feeding nipple 12 defines a mouth leading into an open interior 24 and has a feeding aperture 30 formed therein at the end opposite the mouth, as is common in infant feeding nipples. Preferably, the nipple 12 is formed from non-latex and/or hospital grade materials. The nipple portion 12 may be disposable, intended for a single use, as is common for feeding nipples presently used in hospitals.

The micro-bolus feeder includes an oral syringe 14 having a tip 26 that defines an orifice. The tip end of the syringe 14 is attached to the mouth of the feeding nipple 12 so that the tip 26 of the syringe 14 is in fluid communication with the open interior 24 and feeding aperture 30 of the nipple 12. The syringe 14 is preferably a conventional plunger-type syringe having a tube or barrel 20 and a plunger 22, as is conventionally known. Additionally, graduations 18 or other indicia corresponding to the volume of liquid in the syringe 14 are preferably formed on the barrel 20, allowing the user to properly measure and control the amount of liquid food being fed to infant I. It should be understood that any suitable type of syringe may be utilized, such as a conventional 3 mL or 5 mL syringe or the like.

As shown in FIG. 2, an annular fitting 16 is preferably releasably threaded into the nipple ring 42 of the attachment portion 28 of the system 10. As shown, the annular fitting 16 defines a central, cylindrical passage 32 for removably receiving the syringe 14 to releasably attach the tip end to the mouth of the nipple 12. As described above, any suitable size syringe may be used, such as conventional 3 mL or 5 mL syringes. Annular fittings 16 may be manufactured in a range of sizes having varying sizes of passages 32 to accommodate a variety of syringe sizes by friction or pressure fit. Alternatively, the fitting 16 may be made of a resilient material that allows a single fitting to grip the barrel of syringes of different diameter, or the inner hole of the annular fitting may receive a resilient insert, such as an O-ring, when attached to the syringe of smaller diameter. Threads 34 are formed on the cylindrical outer wall 44 of annular fitting 16 that are dimensioned and configured for releasable mating with the threads 36 formed on the interior of a conventional nipple ring 42 so that any desired nipple 12 can be attached to the syringe.

Preferably, the annular fitting 16 is formed of a transparent, elastomeric or resilient material, which is both waterproof and made from non-latex and/or hospital grade materials. The annular fitting 16 is removably and releasably received within the nipple ring 42 to attach the tip end of the syringe 14 to the mouth of the nipple 12 at the entrance to the open interior 24 of the elastomeric infant feeding nipple 12, thus securing the tip end to the mouth of the feeding nipple 12. Liquid food for infant nutrition is received within the syringe 14 and selectively and controllably expelled therefrom through the feeding aperture 30 of the elastomeric infant feeding nipple 12.

In use, the plunger-type syringe 14, combined with the graduations 18 or other indicia, allows for the controlled feeding of the infant. For medically fragile infants, such as premature infants and the like, the amount of food fed to the infant may be critical. The precise amount (e.g., one-tenth to two tenths of a milliliter) is typically determined by a feeding therapist (often a speech/language pathologist) during a video fluoroscopy swallow study. The present micro-bolus feeder for infants may be used during this examination in order to determine the precise volume of food which the infant can safely ingest. Once this initial, relatively small quantity of liquid food has been delivered to the infant, additional food may be expelled from the syringe 14 in a controlled manner.

In addition to the video fluoroscopy swallow study and simple feeding, the micro-bolus feeder for infants may be used for practice or training in feeding for an infant with a swallowing disorder. Such infants typically cannot swallow thin liquid safely from even the slowest of newborn flow nipples/bottles, and the present micro-bolus feeder may be used for both feeding and physical training in swallowing until oral feeding may be considered.

It will be understood that the nipple 12 may have the shape of a standard baby bottle nipple, or may be an orthodontic nipple (a nipple having a bulb that has a flat side). It will further be understood that the feeding aperture 30 may have different shapes or diameters for controlling the flow rate of milk or other liquid nutrients through the nipple 12, depending upon the infant's age and progress with overcoming dysphagia.

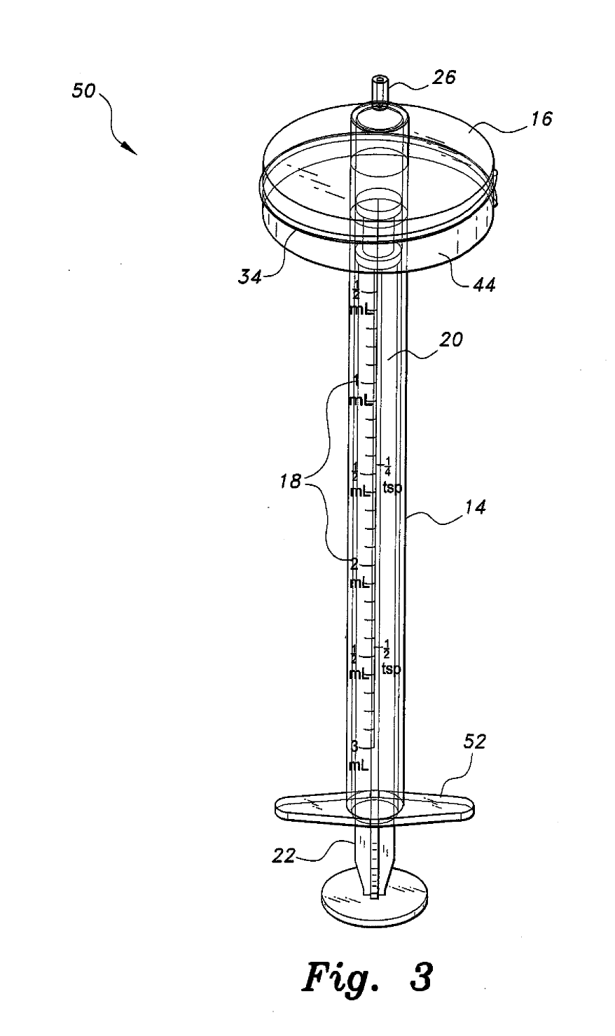

FIG. 3 shows a typical micro-bolus feeder 50 for infants. The feeder 50 includes an oral syringe 14 and the annular adapter fitting 16. Although the annular adapter fitting 16 may be made integral with the syringe 14 as a one-piece, disposable assembly, preferably the fitting 16 is made as a separate component that is releasably attached to the syringe 14 so that a single fitting 16 may be used interchangeably with different syringes 16. The graduated markings 18 on the syringe 14 of FIG. 3 are typical of a 3 mL oral syringe, showing the volume in both milliliters and teaspoons, but a 5 mL syringe may also be used, or less commonly, a 1 mL or a 10 mL syringe may be used.

The syringe 14 has a barrel 20, a plunger 22, a tip 26 defining an orifice, and tabs 52 at the top or plunger end that serve as bearing surfaces for the fingertips when operating the plunger 22. The fitting 16 has an outer cylindrical wall 44 and a helical thread 34 adapted for attaching a nipple ring 42 and nipple 12 to the tip end of the syringe 14.

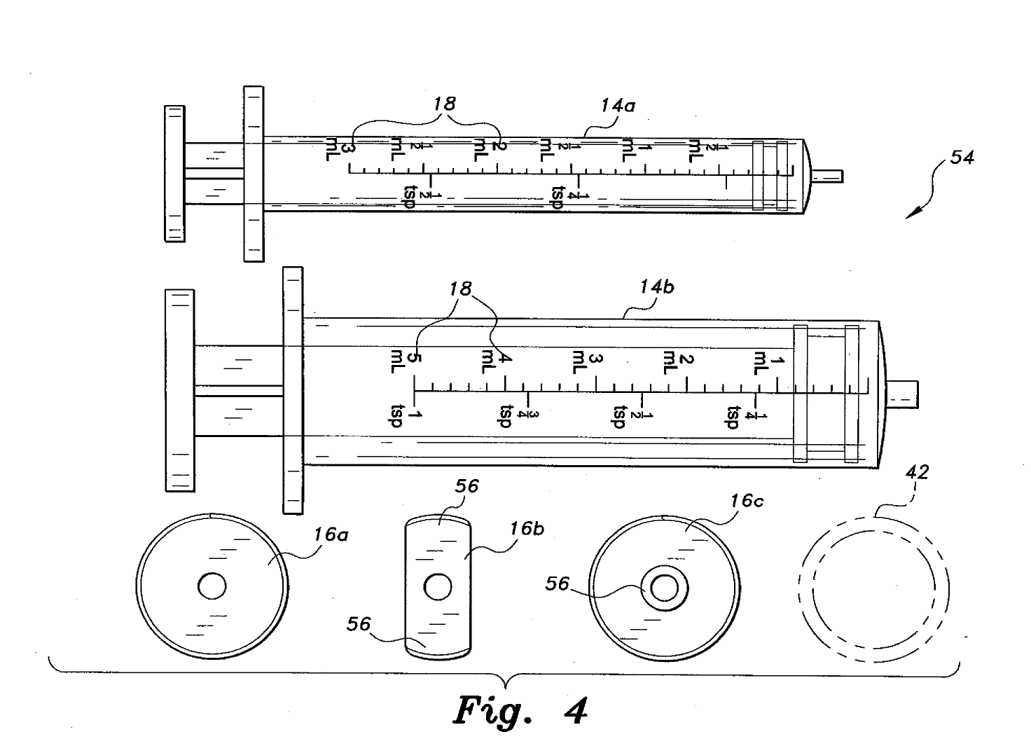

FIG. 4 shows the contents of a typical micro-bolus feeder kit 54 for infants. The kit 54 may include a plurality of disposable oral syringes of different sizes, including a 3 mL oral syringe 14a having graduated markings 18 showing the volume of the syringe 14a to a precision of 1/10 mL, and including a 5 mL oral syringe 14b having graduated markings 18 showing the volume of the syringe 14b to a precision of 2/10 mL . The kit 54 also includes at least one annular adapter fitting. The adapter fitting 16 may be as shown in FIG. 2, having a cylindrical sleeve extending from the center hole of the fitting 16 that forms a pressure fit or friction fit with the barrel 20 of the syringe so that the fitting 16 does not rotate when the nipple ring 42 is threaded onto the fitting 16. Alternatively, as shown in FIG. 4, the annular fitting may be a flat, circular disk 16a without the sleeve; an oval fitting 16b having at least one flattened side (which allows the caregiver to see whether the infant I has swallowed the bolus) and threads only on the arcuate ends 56 that are 180° opposite each other for attaching the nipple ring 42 to the syringe; a circular disk fitting 16c having an insert 58 in the center hole, e.g., the center hole may have a groove defined therein and the insert 58 may be a resilient O-ring lodged in the groove and dimensioned for resiliently gripping the barrel 20, thereby enabling a single fitting to be used alternately with either the 3 mL syringe 14a or the 5 mL syringe 14b, and having an outer peripheral thread for attaching the nipple ring 42 and nipple 12 to the syringe. Optionally, the kit 54 may include a reusable nipple ring 42 that can be used to attach any nipple (standard, orthodontic, etc.) to the syringe.

In use, as described above, the micro-bolus feeder kit for infants provides paced feeding, delivering a pre-determined amount of liquid to the infant, measured in small increments. The graduated syringe 14 allows the user to deliver carefully measured, small amounts of liquid through a feeding device (such as nipple 12, or a spout cup top for older infants, for example). The proper amount of liquid to be delivered to the infant may be determined in advance by fluoroscopy, a bedside evaluation by an expert in infant feeding, or the like.

The micro-bolus feeder kit for infants allows not only delivery of a measured small amount of liquid (1 mL, for example), but also the subsequent opportunity for the infant or child to perform “dry” swallows (i.e., additional swallows to clear the liquid bolus prior to additional liquid being presented). Such a feeding technique may be used in situations where dysphagia is suspected or diagnosed, or for introducing nutritive sucking to an infant who is not yet developmentally ready for traditional bottle feeding. This feeding technique provides a nutritive suck/swallow experience with reduced stress and aspiration risk to an infant who may suffer a risk of aspiration due to a disorganized or immature swallow reflex, a disorganized suck/swallow/breathe triad, respiratory compromise or a history of neurological impairment, for example. Further, usage of the micro-bolus feeder kit for infants allows for consistent nipple placement on the lingual surface (as opposed to pacing by tipping a bottle up and down, thus changing the angle of the nipple on the lingual surface, for example, for providing dry swallows). It should be understood that the micro-bolus feeder kit for infants and the feeding technique described above may be used by clinicians trained in infant and child feeding and swallowing disorders, nurses or other medical professionals providing daily care of an infant, or parents/family members trained in the usage of the micro-bolus feeder kit for infants and its associated feeding technique.

It is to be understood that the present invention is not limited to the embodiments described above, but encompasses any and all embodiments within the scope of the following claims.

I claim:

1. A micro-bolus feeder kit for infants, comprising:
at least one oral syringe having a barrel, a plunger slidable in the barrel, a tip end defining an orifice for ejecting liquid from the barrel, and graduated markings on the barrel corresponding to remaining volume in the barrel between the plunger and the tip end; and
at least one annular adapter fitting having a central aperture and being removably disposed on the barrel of the at least one oral syringe, adjacent the tip end thereof, the at least one annular adapter fitting being dimensioned and configured for attaching a nipple ring thereto for attachment of a nipple over the tip end of the at least one oral syringe for feeding a metered bolus of food to an infant.

2. The micro-bolus feeder kit for infants as recited in claim 1, wherein the at least one annular adapter fitting has an outer peripheral wall and a helical external thread disposed on the outer peripheral wall for threaded engagement with the nipple ring.

3. The micro-bolus feeder kit for infants as recited in claim 2, wherein the at least one annular adapter fitting has a substantially oval contour.

4. The micro-bolus feeder kit for infants as recited in claim 1, wherein the at least one annular adapter fitting comprises a substantially planar disc.

5. The micro-bolus feeder kit for infants as recited in claim 1, further comprising an o-ring received within the central aperture of the at least one annular adapter fitting for resiliently gripping the barrel of the at least one oral syringe.

6. The micro-bolus feeder kit for infants as recited in claim 1, wherein the at least one oral syringe comprises a plurality of oral syringes, each having a different volume.

7. The micro-bolus feeder kit for infants as recited in claim 6, wherein the at least one oral syringe comprises a 3 mL oral syringe and a 5 mL oral syringe.

8. A micro-bolus feeder kit for infants, comprising:
at least one oral syringe having a barrel, a plunger slidable in the barrel, a tip end defining an orifice for ejecting liquid from the barrel, and graduated markings on the barrel corresponding to remaining volume in the barrel between the plunger and the tip end; and
at least one annular adapter fitting having a central aperture and being disposed on the barrel of the at least one oral syringe, adjacent the tip end thereof, the at least one annular adapter fitting being dimensioned and configured for attaching a nipple ring thereto for attachment of a nipple over the tip end of the at least one oral syringe for feeding a metered bolus of food to an infant.

9. The micro-bolus feeder kit for infants as recited in claim 8, wherein the at least one annular adapter fitting has an outer peripheral wall and a helical external thread disposed on the outer peripheral wall for threaded engagement with the nipple ring.

10. The micro-bolus feeder kit for infants as recited in claim 9, wherein the at least one annular adapter fitting has a substantially oval contour.

11. The micro-bolus feeder kit for infants as recited in claim 8, wherein the at least one annular adapter fitting comprises a substantially planar disc.

12. The micro-bolus feeder kit for infants as recited in claim 8, further comprising an o-ring received within the central aperture of the at least one annular adapter fitting for resiliently gripping the barrel of the at least one oral syringe.

13. The micro-bolus feeder kit for infants as recited in claim 8, wherein the at least one annular adapter fitting is removably disposed on the at least one oral syringe.

14. The micro-bolus feeder kit for infants as recited in claim 13, wherein the at least one oral syringe comprises a plurality of oral syringes, each having a different volume.

15. The micro-bolus feeder kit for infants as recited in claim 14, wherein the at least one oral syringe comprises a 3 mL oral syringe and a 5 mL oral syringe.

16. The micro-bolus feeder kit for infants as recited in claim 8, further comprising:
a nipple ring for attachment to the at least one annular adapter fitting; and
a nipple for attaching to the nipple ring.

17. A method for feeding an infant a pre-determined amount of liquid food, comprising:
providing a micro-bolus feeder kit, the micro-bolus feeder kit including an oral syringe having a barrel; a plunger slidable in the barrel; a tip end defining an orifice for ejecting liquid from the barrel; graduated markings on the barrel corresponding to remaining volume in the barrel between the plunger and the tip end; an annular adapter fitting having a central aperture and being removably disposed on the barrel of the oral syringe, adjacent the tip end thereof; a nipple ring attached to the annular adapter fitting; and a nipple attached to the nipple ring and disposed over the tip end of the oral syringe for feeding a metered bolus of food to an infant;
dispensing a liquid food in the oral syringe; and
expelling at least a portion of the liquid food from the oral syringe through the nipple.

18. The micro-bolus feeder kit for infants as recited in claim 17, wherein the annular adapter fitting has an outer peripheral wall and a helical external thread disposed on the outer peripheral wall for threaded engagement with the nipple ring.

19. The micro-bolus feeder kit for infants as recited in claim 18, wherein the annular adapter fitting has a substantially oval contour.

20. The micro-bolus feeder kit for infants as recited in claim 17, wherein the annular adapter fitting comprises a substantially planar disc.
